(19) United States
(12) Patent Application Publication
(10) Pub. No.: US 2007/0293946 A1
(43) Pub. Date: Dec. 20, 2007

(54) Middle Turbinate Medializer

(75) Inventors: Donald Albert Gonzales, San Antonio, TX (US); Michael Charles Larson, Colorado Springs, CO (US); Fred B. Dinger III, San Antonio, TX (US); Gabriele G. Niederauer, San Antonio, TX (US); Jeffrey S. Wrana, San Antonio, TX (US)

Correspondence Address: FULBRIGHT & JAWORSKI L.L.P., 600 CONGRESS AVE., SUITE 2400, AUSTIN, TX, 78701, US

(73) Assignee: ENTRIGUE SURGICAL, INC.

(21) Appl. No.: 11/747,442

(22) Filed: May 11, 2007

Related U.S. Application Data
(60) Provisional application No. 60/800,176, filed on May 12, 2006.

(51) Int. Cl. A61F 2/18

(52) U.S. Cl. 623/10

(57) ABSTRACT

Medializing the middle turbinate in the nose has been realized as a solution to the common complication of adhesions following nasal and sinus surgery. The invention provides a system for medializing the middle turbinate by attaching the middle turbinate temporarily to the nasal septum. The attachment is performed using a wafer with means on both sides for attaching the wafer to a mucosal surface. The attachment may also be performed using a tissue adhesive, pins, or other medical devices described herein. The invention also provides a system for attaching the uvula to the nasopharyngeal side of the soft palate. The invention provides a medical device for use in the inventive procedures as well as methods for the procedures and kits for use by a physician.

### RELATED APPLICATIONS

The present application claims priority under 35 U.S.C. § 119(e) to U.S. provisional patent application, U.S. Ser. No. 60/800,176, filed May 12, 2006; which is incorporated herein by reference.

### BACKGROUND OF THE INVENTION

Sinusitis is a progression of inflammation, stasis, infection, and continued inflammation. Typically, the beginning of all sinus infections is either allergy or viral infection. Both of these conditions lead to swelling of the sinus and nasal mucosa that when severe enough, causes the small holes, called ostia, of the sinuses to close. Once the ostia is closed, the environment inside the sinuses, specifically the maxillary sinus, becomes conducive to bacterial growth. The way this typically occurs is that once the ostia is shut, the oxygen content of the sinus drops and the fluid inside the sinus is unable to escape which leads to further inflammation. The reduced oxygen content and inflammation disrupts the ability of the cilia of the cells of the sinus to operate properly which leads to further stasis.

The typical patient that is seen by the otolaryngologist is started on antibiotics. Usually the antibiotic course can be as long as six weeks to eradicate the bacteria and bring the sinuses back to normal. For those patients in whom antibiotics do no relieve the problem, the only alternative is surgery. Although sinus and nasal surgeries are now common with 500,000 to 700,000 of such surgeries being performed annually in the U.S., these surgeries are typically both destructive and permanent. Around 10% of patients who undergo sinus surgery have scarring that leads to continued sinus problems which frequently require revision surgery.

One frequent problem is postoperative adhesions. These adhesions occur between the middle turbinate and the adjacent nasal areas. One particular problem is the adhesion of the middle turbinate to the lateral nasal wall. Some surgeons have proposed removing the lower half of the middle turbinate to avoid this problem. This procedure, however, has its own problems (e.g., crust formation, nasal hygiene issues).

Other solutions that have been suggested include placing a suture through the middle turbinate on one side of the nose, through the nasal septum, and then through the middle turbinate on the other side before the suture is tied off. Such a suture draws the middle turbinates medially and prevents the formation of adhesions between the middle turbinate and the lateral nasal wall. However, this suture is difficult and time-consuming to place and requires the puncturing of three separate structures in the nose. This can lead to discomfort for the patient, bleeding, infection, and other complications.

Another solution surgeons have proposed is the use of various packing materials and splints. The use of these materials and devices however leads to the formation of scar tissue, which is undesirable and can lead to airway obstruction and infection. The adhesion of the middle turbinate to adjacent structures in the nose remains a problem in nasal and sinus surgery.

Given this serious and common complication of sinus surgery, there remains a need in the art for preventing the formation of adhesions between the middle turbinate and adjacent nasal structures, particularly the lateral nasal wall. The desired solution preferably limits or eliminates the complications of the other proposals which have been used including infection, scar tissue formation, adhesions, bleeding, and patient discomfort.

### SUMMARY OF THE INVENTION

The present invention provides a system for reducing the adhesions formed in a patient's nasal cavity following a sinus or nasal procedure. In particular, the inventive system reduces the formation of adhesions between the lateral nasal wall and the middle turbinate by attaching the middle turbinate to the nasal septum. This system pulls the middle turbinate medially to avoid the formation of adhesions which may lead to further complications after sinus or nasal surgery. The attachment of the middle turbinate to the nasal septum may be temporary or permanent. This system may also be used prior to surgery to pull the middle turbinate away from the uncinate process to make surgeries in this area easier.

In one aspect, the invention provides a medical device for medializing the middle turbinate. As shown in FIGS. 1 and 2, in certain embodiments, the device is a wafer with a means for attaching the wafer to a surface (e.g., a mucosal surface) on both sides of the wafer. The means for attaching may include a tissue glue (e.g., cyanoacrylate, fibrin sealant), hooks, barbs, pins, staples, arrows, etc. The wafer thereby can bring two structures together. The device is particularly useful in attaching the middle turbinate to the nasal septum thereby preventing the formation of adhesions between the middle turbinate and the lateral nasal wall which can lead to complications after nasal and sinus surgeries. The wafer can be any shape including discs, rings, triangular-shaped wafers, polygonal-shaped wafers, zig-zag, etc. In certain instances, the wafer may include contours to fit comfortably inside the nose of the patient. For example, the wafer may include a contour for the middle turbinate on one side and be flat on the side that abuts the nasal septum. The wafer is typically approximately 1 cm by approximately 1 cm so that it can rest comfortably inside the nose of the patient between the middle turbinate and nasal septum. The device is approximately 0.75 mm or less in thickness. The wafer may be made from any biocompatible material.

In another embodiment, the device comprises a sling-like portion to securely grasp the turbinate and barbs, adhesives, or other fixation means for attaching the device with the turbinate to the nasal wall. In yet another embodiment, the device is an arrow-like device or pin used to fix the middle turbinate to the nasal wall by pinning the turbinate. See, e.g., FIGS. 5-9. Such devices or pins may have protrusions, flanges, barbs, coatings, or bumps on their surfaces to prevent the device from falling out. See, e.g., FIGS. 7-9.

Preferably, the wafer or other device is made from a bioabsorbable material, for example, a PLGA co-polymer. Therefore, after the patient's nose has healed, the wafer or other device is absorbed by the body, thus avoiding the permanent attachment of the middle turbinate to the nasal septum. In certain embodiments, the wafer or other device is made of a non-bioresorbable material; thus, the device, if needed, can be removed later or left in place permanently.

In another aspect, the invention provides a method for medializing the middle turbinate. In certain embodiments, the wafer as described above is inserted into the nose of patient between the middle turbinate and the nasal septum, and pressure is applied to the middle turbinate and nasal septum to attach these two structures via the wafer. In another embodiment, tissue adhesive (e.g., a cyanoacrylate adhesive) rather than the inventive wafer is used to adhere the middle turbinate to the nasal septum. In still another embodiment, the wafer may be used in conjunction with a tissue adhesive. In still other embodiments, the middle turbinate is pinned to the nasal septum. In yet other embodiments, the sling-like device is used to draw the middle turbinate toward the nasal septum. By any of these approaches, the middle turbinate is adhered to the nasal septum thereby moving the middle turbinate medially. The method is typically performed during a nasal or sinus procedure or surgery (e.g., endoscopic sinus surgery). The device may be implanted at the beginning of a procedure to pull the middle turbinate away from the uncinate process to make the procedure easier. This may move the middle turbinate out of the way for better visualization of the lateral wall and such structures as the ostia leading to the paranasal sinuses and the uncinate process. The device may then be left in place to prevent the formation of adhesions between the middle turbinate and the nasal septum. The wafer or other device may be implanted using medical devices for endoscopic surgery or may be implanted using specially designed tools for using the device. After the device is implanted or adhesive is applied, it typically stays in place long enough for the mucosa of the nasal passage to heal. The device or adhesive may stay in place for a time ranging from 1 week to 6 months. Once the mucosa has healed and there is no longer a risk of adhesions forming, the device may be removed or be absorbed by the patient's body. The device may also fall out of place, be swallowed by the patient along with mucus, and be safely degraded by the digestive system of the patient.

In certain embodiments, the invention provides a method for medializing the middle turbinate using a tissue glue (e.g., cyanoacrylate, fibrin sealant) alone. Tissue glue is applied to the middle turbinate and/or the nasal septum, and pressure is applied to these two structures so that they come in contact for a sufficient time for the tissue glue to set. The adhesion of the middle turbinate to the nasal septum allows for the healing of the nasal mucosa without the risk of adhesions developing between the middle turbinate and the lateral nasal wall. Over time, the tissue glue breaks down, and the middle turbinate is subsequently released from the nasal septum. In the case of using a tissue glue such as cyanoacrylate alone, the glue may need to be reapplied by the treating physician every week or as needed until the mucosa heals and there is limited risk of adhesions forming.

In another aspect, the invention provides a method of using the inventive device or tissue adhesive to attach the uvula to the nasopharyngeal side of the soft palate. Such a procedure is illustrated in FIG. 3. The inventive procedure is particularly useful in treating snoring or sleep apnea. The attachment may be permanent or temporary as needed.

The invention also provides an instrument for inserting the inventive medical device into the nose of a patient. The instrument typically includes a comfortable grip and an elongated end with a means for holding and releasing the inventive medical device in place. The invention also provides an instrument for applying pressure to the middle turbinate and nasal septum around the medical device in order to attach the middle turbinate to the nasal septum by means of the medical device. An example of an instrument for inserting the inventive wafer is shown in FIG. 4.

In another aspect, the invention provides a kit including the inventive medical device. The kit may also include tissue glue (e.g. cyanoacrylate, fibrin sealant), pharmaceutical agents (e.g., steroids, non-steroidal anti-inflammatory agents, antibiotics), an instrument for inserting the inventive medical device and attaching the middle turbinate to the nasal septum, an instrument for removing the inventive device, instructions for inserting the inventive medical device, etc. Typically, these items are conveniently packaged for the use by a treating physician. In certain embodiments, the items are sterilely packaged.

The present invention fills a need in nasal and sinus surgery for preventing adhesions after surgery by temporarily adhering the middle turbinate to the nasal septum. After the nasal mucosa has healed sufficiently the attachment naturally breaks down or is manually removed, thereby restoring the natural anatomy of the nasal passage. The inventive system reduces the complications following sinus and nasal surgery.

### BRIEF DESCRIPTION OF THE DRAWING

FIG. 1 shows an example of the inventive wafer-like medical device with barbs for attaching to the nasal mucosa of the septum and the mucosa of the middle turbinate.

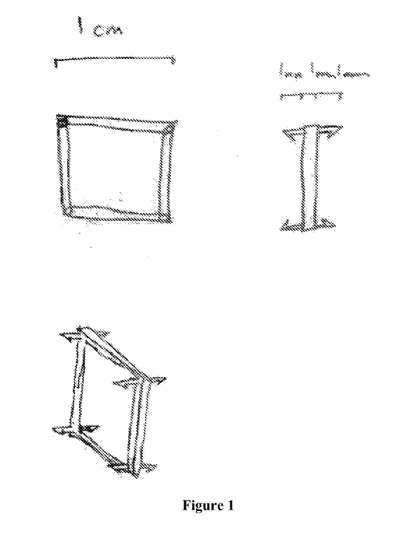

FIG. 2 shows the placement of the inventive device and the resulting medialization of the middle turbinate.

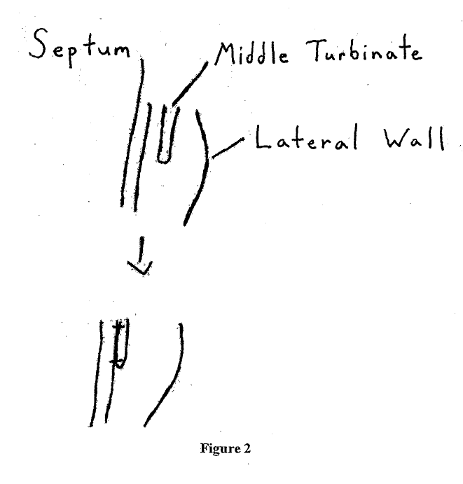

FIG. 3 shows the use of an inventive medical device with barbs to attach the uvula to the nasopharyngeal side of the soft palate.

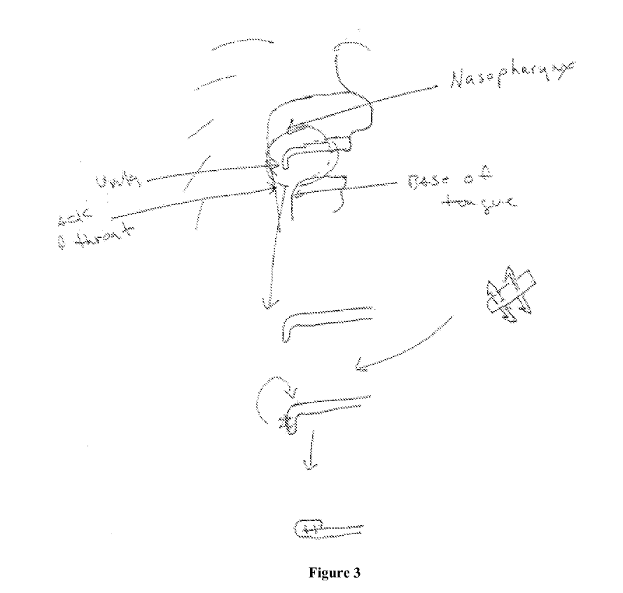

FIG. 4 is an illustration of an instrument for placing the inventive wafer for attaching the middle turbinate to the nasal septum.

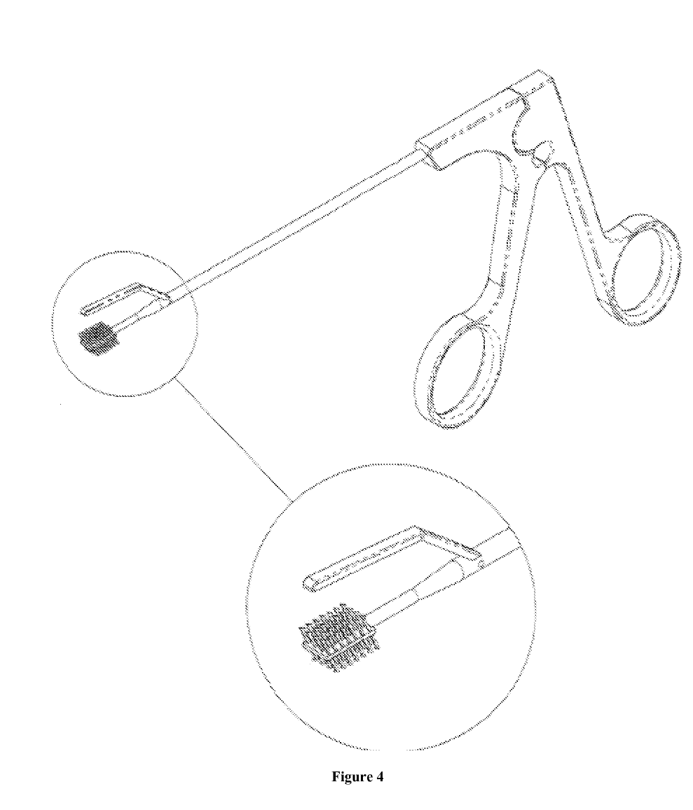

FIG. 5 shows exemplary pins for attaching the nasal mucosa of the septum and the mucosa of the middle turbinate.

FIG. 6 shows another design of the inventive pins that have ridges on the pointed tip.

FIG. 7 shows another design of the inventive pins with protrusions for preventing the pin from dislodging.

FIG. 8 shows another design of the inventive pins with bump-like protrusions.

FIG. 9 shows another design of the inventive pins with barbs.

FIG. 10 shows a wafer with barbs for attaching the nasal mucosa of the septum to the mucosa of the middle turbinate.

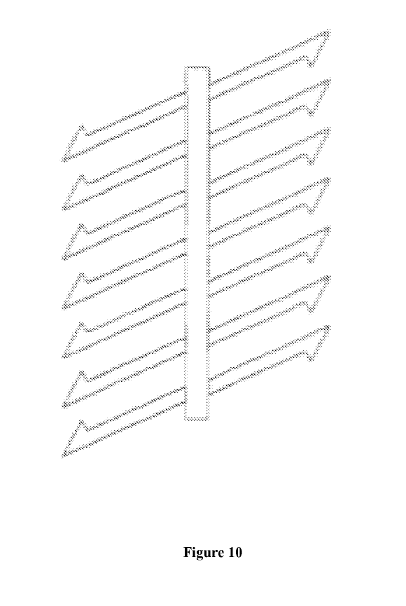

FIG. 11 shows a circular design with barbs for attaching the nasal mucosa of the septum to the mucosa of the middle turbinate.

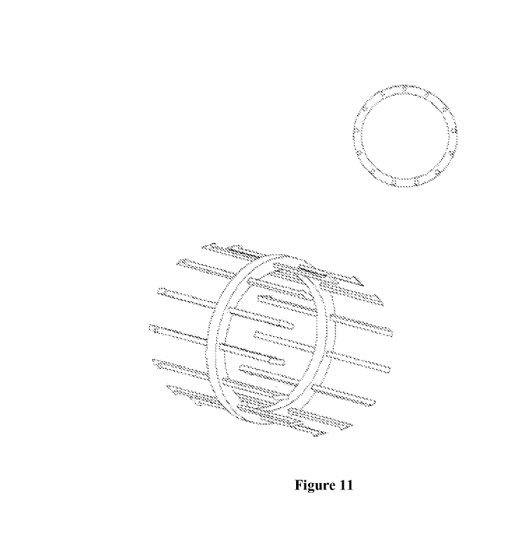

FIG. 12 shows a zig-zag design of the inventive medical device.

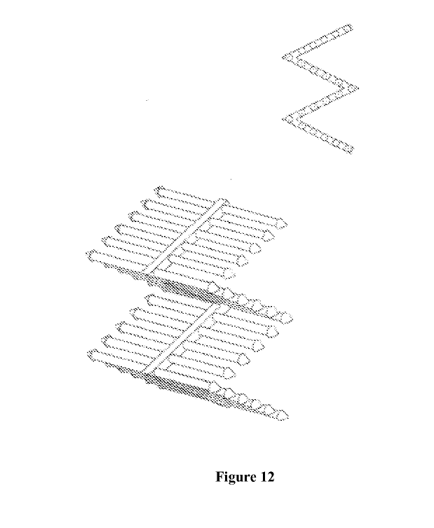

FIG. 13 shows a side view of an exemplary inventive medical device.

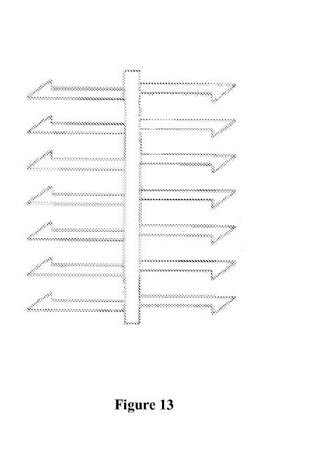

FIG. 14 shows a side view of another exemplary inventive medical device with curved barbs.

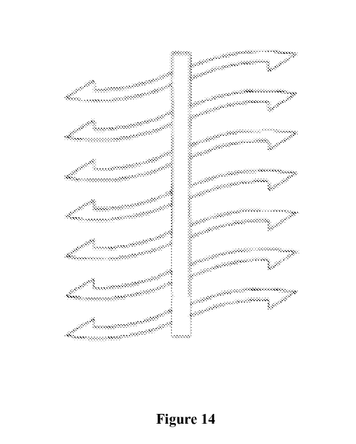

FIG. 15 shows a side view of another exemplary inventive medical device with curved barbs.

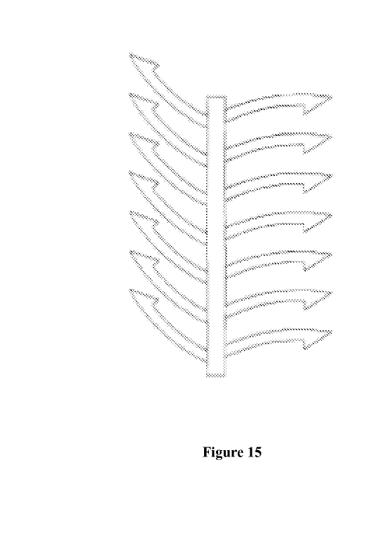

FIG. 16 shows a side view of another exemplary inventive medical device with curved barbs.

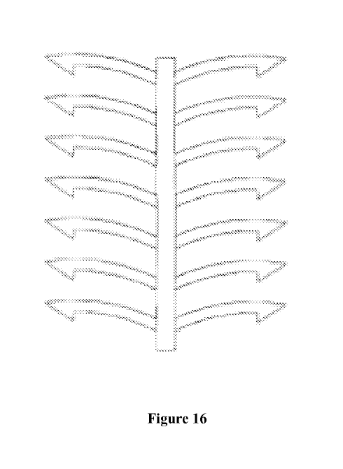

FIG. 17 shows a side view of another exemplary inventive medical device with slanted barbs with respect to the surface of the wafer.

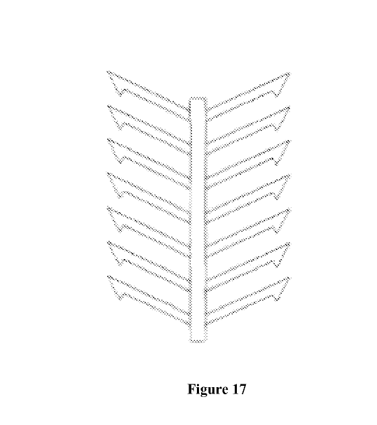

FIG. 18 shows another design for the inventive medical device with two barbs for attachment.

FIG. 19 shows a design with four barbs.

FIG. 20 shows a planar design for the inventive medical device.

FIG. 21 shows a sling-type device in which the sling portion is slipped around the turbinate and then the device is secured to the nasal wall with piercing arrows or barbs.

### DETAILED DESCRIPTION OF THE INVENTION

The present invention provides a system for medializing the middle turbinate following and/or during nasal or sinus surgery. The invention stems from the recognition that attaching the middle turbinate to the nasal septum, thereby drawing the middle turbinate medially would prevent the formation of adhesions between the middle turbinate and lateral wall. These adhesions are known to cause further complications post surgery including paranasal sinus blockage. The inventive system prevents the formation of adhesions between the middle turbinate and the lateral nasal wall and therefore the subsequent complications. These adhesions frequently require post-revision surgery to remove the adhesions. The invention not only provides a medical device for use in medializing the middle turbinate but also provides kits, instruments for placing and removing the inventive devices, and procedures for medializing the middle turbinate.

A patient suffering from nasal or sinus disease (e.g., allergies, infection) having undergone a sinus or nasal procedure is at a substantial risk of developing adhesions between various structures in the nasal passage due to trauma to the mucosal surfaces. In order to prevent the formation of adhesions, particularly between the lateral nasal wall and the middle turbinate, the middle turbinate is attached at least temporarily to the nasal septum. In certain embodiments, the middle turbinate is attached to the nasal septum prior to starting the procedure or surgery in order to make the surgery easier. The attachment can then be left in place after the procedure or surgery is concluded. This attachment is accomplished using a medical device such as a wafer or pin with means for attaching middle turbinate to the nasal septum or using a tissue glue such as a cyanoacrylate adhesive, fibrin sealant, or other natural or synthetic adhesive. In most instances, the attachment is temporary. Typically, the attachment is only in place for the length of time needed for the nasal mucosa to heal. Once the mucosa is healed, the chance of adhesions forming is greatly reduced. The attachment may be manually severed, or the means for attaching the middle turbinate and the nasal septum may degrade over time. For example, the device may be absorbed by the patient's body. The device may fall out of place and be harmlessly swallowed by the patient and degraded in the patient's digestive system. Or the adhesive may break down releasing the middle turbinate from the nasal septum.

The attachment whether by medical device or adhesive alone may last from 1 week to 24 months depending on the judgment of the treating physician. In certain embodiments, the attachment lasts from 2 weeks to 8 weeks, or 3 weeks to 6 weeks. In other embodiments, the attachment lasts for approximately 1 month, 2 months, 3 months, 4 months, 5 months, or 6 months. In other embodiments, the attachments last for approximately 9 months, 12 months, 18 months, or 24 months. If longer attachment is necessary, the inventive procedure may be repeated once, twice, three times, or more depending upon the patient and the judgment of the treating physician. In certain embodiments where a tissue adhesive alone is used, the adhesive may need to be reapplied every few days, every week, every two weeks, or as needed until the nasal mucosa is healed. In certain embodiments where a cyanoacrylate adhesive is used, the adhesive is reapplied approximately every week.

As described above for drawing medially the middle turbinate, the inventive device may also be used to attach the uvula to the nasopharyngeal side of the soft palate. Such an attachment is particularly useful in patients who snore or patients who suffer from sleep apnea. The attachment may also be used to move the uvula out of the way for a procedure involving the oronasopharynx. The attachment may be temporary or permanent. The wafer or other medical device as described herein is inserted into the oronasopharynx of the patient either through the nose or mouth. The device is then used to attach the soft palate to the uvula. Pressure may be applied to the uvula and soft palate to attach these two structures via the device. In one particular embodiment, tissue adhesive (e.g., a cyanoacrylate adhesive) rather than an inventive device is used to adhere the uvula to the nasal septum. In still another embodiment, an inventive device may be used in conjunction with a tissue adhesive. The method is typically performed during a procedure or surgery. The device may be implanted using medical devices for endoscopic surgery or may be implanted using specially designed tools for using the device.

As will be appreciated by those of skill in the art, the inventive system may be used in attaching other structures in the body to each other (e.g., in the oronasopharynx, gastrointestinal system, genitourinary system, etc.). In certain embodiments, the system is used in the oronasopharynx and attached to one or more of the following structures: turbinates, nasal septum, uvula, hard palate, soft palate, tonsils, tongue, gingiva, epiglottis, walls of the sinus, and sides of the oral cavity. The inventive system is particularly useful in attaching mucosal surfaces. In certain embodiments, the inventive system is not used to approximate wound surfaces. In other embodiments, the inventive system is used to approximate wound surfaces.

In one embodiment, the medical device is a thin wafer with both sides of the wafer having means for attaching the wafer to a surface. Therefore, the wafer can be used to bring two structures such as the middle turbinate and the nasal septum together. The wafer can be any shape or size capable of being placed into the space between the middle turbinate and nasal septum of a patient, preferably a human patient. In certain embodiments, the wafer is circular. In other embodiments, the wafer is triangular shaper, rectangular shaped, or polygonal shaped. In yet other embodiments, the wafer is a ring. In certain embodiments, the wafer is a zig-zag shape. The surface area of the sides of the wafer should provide a large enough surface area to adequately attach to the middle turbinate and nasal septum so that the middle turbinate can be pulled medially. The wafer is typically approximately 0.2 cm-2 cm in length by approximately 0.2 cm-2 cm in width. In certain embodiments, the length ranges from approximately 0.5 cm to approximately 1.5 cm. In certain embodiments, the length ranges from approximately 1 cm to approximately 2 cm. In certain embodiments, the length ranges from approximately 1.5 cm to approximately 2 cm. In certain embodiments, the length ranges from approximately 0.25 cm to approximately 0.75 cm. In certain embodiments, the length ranges from approximately 0.5 cm to approximately 1 cm. In certain embodiments, the width ranges from approximately 0.5 cm to approximately 1.5 cm. In certain embodiments, the width ranges from approximately 1 cm to approximately 2 cm. In certain embodiments, the width ranges from approximately 1.5 cm to approximately 2 cm. In certain embodiments, the width ranges from approximately 0.25 cm to approximately 0.75 cm. In certain embodiments, the width ranges from approximately 0.5 cm to approximately 1 cm. In certain embodiments, the wafer is approximately 1.5 cm by approximately 1.5 cm. In certain embodiments, the wafer is approximately 1 cm by approximately 1 cm. In certain embodiments, the wafer is approximately 0.75 cm by approximately 0.75 cm. In certain embodiments, the wafer is approximately 0.5 cm by approximately 0.5 cm. In certain embodiments, the wafer is approximately 0.25 cm by approximately 0.25 cm. For pediatric patients, the wafer may be smaller, that is, less than 1 cm by 1 cm. Also, the wafer may be smaller where more than one wafer is being used to attach the middle turbinate to the nasal septum. The wafer is approximately 0.75 mm in thickness; however, the thickness of the wafer may vary from less than 0.2 mm to approximately 0.5 cm. In certain embodiments, the thickness of the wafer is in the range of approximately 0.5 mm to approximately 1.5 mm. In other embodiments, the wafer is a thin film of less than 0.2 mm in thickness.

The means on the wafer or other device described herein for attaching the device to a surface such as the surface of the middle turbinate or the surface of the nasal septum include any chemical adhesive or mechanical means of forming an attachment. The means for attaching is preferably suitable for attaching the device to a mucosal surface. In certain embodiments when a chemical adhesive is used, the adhesive is a cyanoacrylate adhesive. In other embodiments, a similar synthetic glue is used as the adhesive. In other embodiments when an adhesive is used, the adhesive is a fibrin sealant or other natural substance such as mussel adhesive protein, frog glue, etc. These adhesives have been shown useful in closing wounds and are commercially available. The adhesive may be applied to the device immediately before implanting the device in the patient. Mechanical means for forming an attachment include pins, staples, rivets, barbs, or hooks on the surface of the device which allow attachment to a surface. The surface of the wafer or other device may also be constructed to have a fibrous surface similar to Velcro® for attaching the device to a tissue such as one with a mucosal surface. These attachment means typically extend less than approximately 1 cm from the surface of the wafer or other device, more preferably, less than 0.5 cm from the surface of the device. In certain embodiments, they extend less than 1 mm from the surface. Usually multiple pins, staples, rivets, barbs, or hooks are used to provide a secure attachment. These means typically do not puncture through the entire nasal structure. In certain embodiments, the mechanical means only penetrate the mucosa. In certain embodiments, an adhesive (e.g., cyanoacrylate, fibrin sealant, mussel adhesive protein, frog glue) is used in conjunction with a mechanical means for attachment.

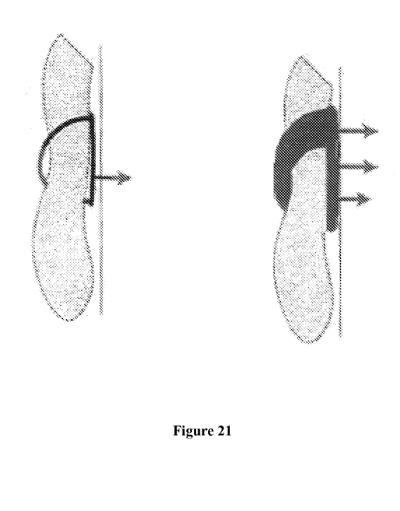

In another embodiment, the device comprises a sling-like or pouch-like portion that is slipped around the middle turbinate and barbs or arrows for securing the device to the nasal septum. The device thereby draws the middle turbinate medially toward the nasal septum. The sling portion may be made of a thin suture-like material, or it may be made of a wider material, which is solid or mesh-like. An illustration of such a device is shown in FIG. 21.

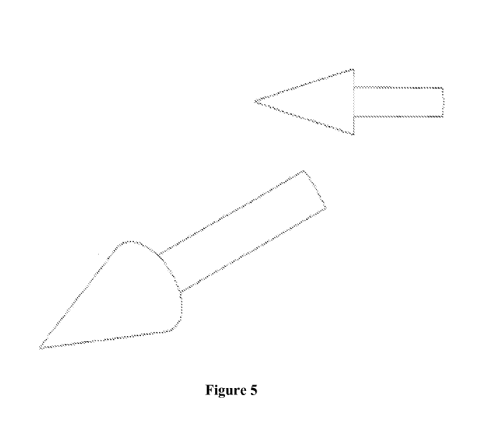

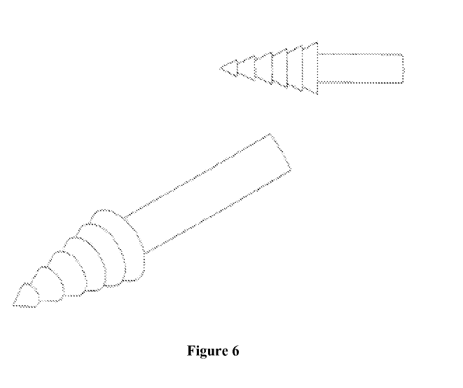

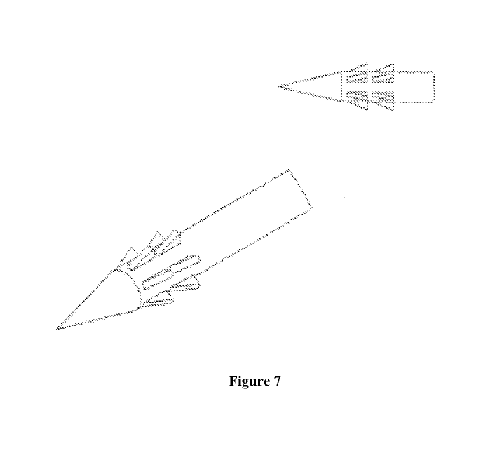

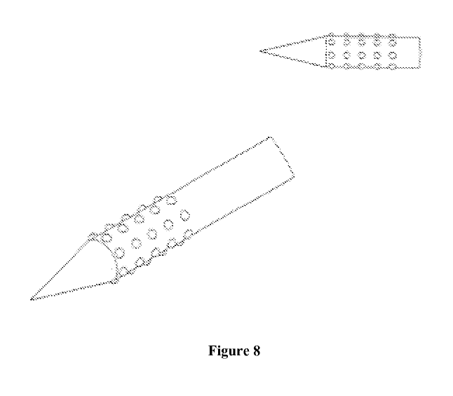

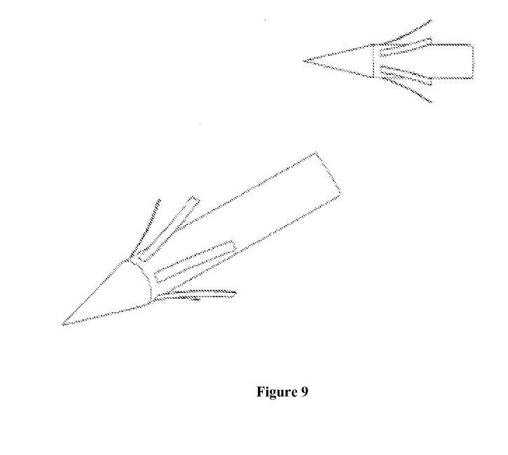

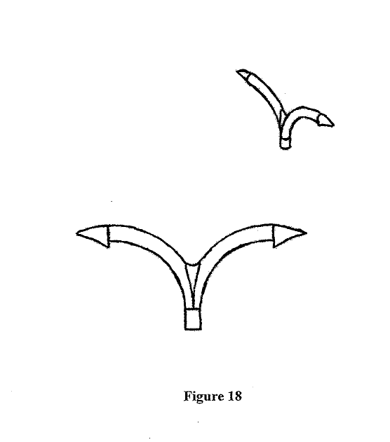

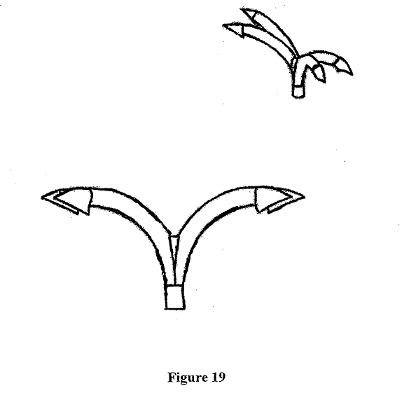

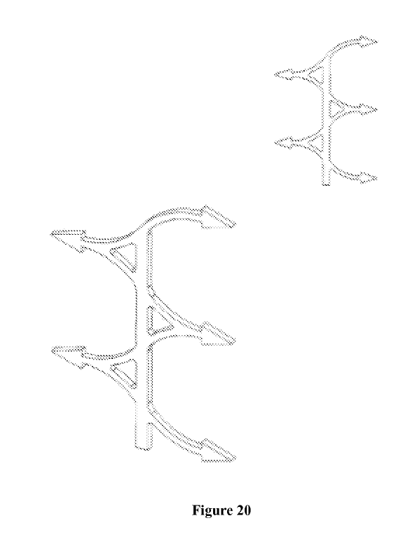

In yet another embodiment, the device is a pin for attaching the middle turbinate to the nasal septum. These devices are typically less than 2 cm in length. In certain embodiments, the devices are approximately 0.5 cm to 1.5 cm in length. In certain embodiments, the devices are approximately 0.25 cm, approximately 0.5 cm, approximately 0.75 cm, approximately 1 cm, approximately 1.25 cm, approximately 1.5 cm, approximately 1.75 cm, or approximately 2 cm in length. The surface of the pin may include protrusions to prevent the pin from coming out. The protrusions may be small barbs, bumps, ridges, etc. The pin may also be coated to prevent the pin for easily dislodging. The pin may also be coated to make it more biocompatible or allow for release of a bioactive agent. Exemplary designs for such pin devices are shown in FIGS. 5-9. Other devices with two or more pins are also included within the invention as shown in FIGS. 18-20. Such devices may be smaller than the wafer devices.

Any of the inventive devices can be made of any biocompatible material. Preferably, the device is made of a biodegradable material. In certain embodiments, the material is a biodegradable polymer. The material may be synthetic (e.g., polyesters, polyanhydrides) or natural (e.g., proteins, rubber, polysaccharides). Preferably, the device is made of a biodegradable material. In certain embodiments, the material is a biodegradable polymer. In certain embodiments, the material is a homopolymer. In certain embodiments, the material is a co-polymer. In other embodiments, the material is a block polymer. In other embodiments, the material is a branched polymer. In other embodiments, the material is a cross-linked polymer. In certain embodiments, the polymer is a polyester, polyurethane, polyvinyl chloride, polyalkylene (e.g., polyethylene), polyolefin, polyanhydride, polyamide, polycarbonate, polycarbamate, polyacrylate, polymethacrylate, polystyrene, polyurea, polyether, polyphosphazene, poly(ortho esters), polycarbonate, polyfumarate, polyarylate, polystyrene, or polyamine. In certain embodiments, the polymers is polylactide, polyglycolide, polycaprolactone, polydioxanone, polytrimethylene carbonate, and co-polymers thereof. Polymers that have been used in producing biodegradable implants and are useful in preparing the inventive devices include alpha-polyhydroxy acids; polyglycolide (PGA); copolymers of polyglycolide such as glycolide/L-lactide copolymers (PGA/PLLA), glycolide/D,L-lactide copolymers (PGA/PDLLA), and glycolide/trimethylene carbonate copolymers (PGA/TMC); polylactides (PLA); stereocopolymers of PLA such as poly-L-lactide (PLLA), poly-D,L-lactide (PDLLA), L-lactide/D,L-lactide copolymers; copolymers of PLA such as lactide/tetramethylglycolide copolymers, lactide/trimethylene carbonate copolymers, lactide/δ-valerolactone copolymers, lactide ε-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA); poly-p-dioxanone (PDS); poly-δ-valerolatone; poly-r-caprolactone; methylmethacrylate-N-vinyl pyrrolidone copolymers; polyesteramides; polyesters of oxalic acid; polydihydropyrans; polyalkyl-2-cyanoacrylates; polyurethanes (PU); polyvinyl alcohol (PVA); polypeptides; poly-beta-maleic acid (PMLA); poly(trimethylene carbonate); poly(ethylene oxide) (PEO); poly(β-hydroxyvalerate) (PHVA); poly(ortho esters); tyrosine-derived polycarbonates; and poly-beta-alkanoic acids. In certain embodiments, the polymer is a polyester such as poly(glycolide-co-lactide) (PLGA), poly(lactide), poly(glycolide), poly(D,L-lactide-co-glycolide), poly(L-lactide-co-glycolide), poly-β-hydroxybutyrate, and polyacrylic acid ester. In certain embodiments, the device is made of PLGA. In certain embodiments, the device is made of 85% D,L-lactide and 15% glycolide co-polymer. In certain embodiments, the device is made of 50% D,L-lactide and 50% glycolide co-polymer. In certain embodiments, the device is made of 65% D,L-lactide and 35% glycolide co-polymer. In certain embodiments, the device is made of 75% D,L-lactide and 25% glycolide co-polymer. In certain embodiments, the device is made of 85% L-lactide and 15% glycolide co-polymer. In certain embodiments, the device is made of 50% L-lactide and 50% glycolide co-polymer. In certain embodiments, the device is made of 65% L-lactide and 35% glycolide co-polymer. In certain embodiments, the device is made of 75% L-lactide and 25% glycolide co-polymer. In certain embodiments, the device is made of poly(caprolactone). In certain embodiments, the device is made of Pebax, Polyimide, Braided Polyimide, Nylon, PVC, Hytrel, HDPE, or PEEK. In certain embodiments, the device is made of a fluoropolymer such as PTFE, PFA, FEP, and EPTFE. In certain embodiments, the device is made of latex. In other embodiments, the device is made of silicone. The polymer typically has a molecular weight sufficient to be shaped by molding or extrusion. The device is typically made of a material that is bioabsorbed after the device is not longer needed. For example, the device may degrade after 1 week, 2 weeks, 3 weeks, 1 month, 2 months, 3 months, 4 months, 5 months, 6 months, 9 months, 1 year, 1.5 years, 2 years, 3 years, etc. The polymer used to make the device may be selected based on its degradation profile. As would be appreciated by one of skill in this art, the composition of the wafer may be varied to achieve the desired lifetime in vivo of the wafer.

In other embodiments, the device is made of a metal. In other embodiments, the device is made of an alloy. In certain embodiments, the device is made of stainless steel. In certain embodiments, the device is made of a magnesium alloy (e.g., magnesium based alloy AE21). See, e.g., Heublein et al., “Biocorrosion of magnesium alloys: a new principle in cardiovascular implant technology?” Heart 89:651-56, 2003; incorporated herein by reference. In certain embodiments, the device is made of titanium. In certain embodiments, the device is made of a titanium alloy. In certain embodiments, the device is made of a superelastic alloy such as Nitinol. Metal devices may be optionally coated with a biocompatible coating. In the case where the device is made of a metal, the device may be inserted permanently or may be removed manually after the device is no longer needed.

The device may be coated with a biocompatible material. In certain embodiments, the device is made of or is coated with a timed-release formulation of a pharmaceutical agent. For example, a steroid, analgesic, anti-inflammatory agent, or antibiotic may be released by the wafer. In certain embodiments, the device is coated with a bioactive agent. Bioactive agents include small molecules, drugs, polynucleotide, proteins, peptides, etc. In certain embodiments, the bioactive agent may promote wound healing. In certain embodiments, the bioactive agent stimulates the formation of a desired tissue. In certain embodiments, the bioactive agent accelerates the integration of the turbinate with the nasal septum. In yet other embodiments, the tube may be coated with a material to prevent cell growth such as a cytotoxic agent. The device may also be coated with a substance to prevent the formation of adhesions. For example, the device may be coated with a polysaccharide such as hyaluronate. The device may also be coated with a polymeric coating such as Teflon.

The inventive medical device may be packaged in kits for convenience. In certain embodiments, the kits may also include all or some of the following items: an instrument for implanting the device, an instrument for removing the device, adhesive, pharmaceutical agents, nasal sprays, gauze, bandages, disinfectant, and instructions for using the device. In certain embodiments, the kits are sterilely package for convenient use by a surgeon or other medical professional.

What is claimed is:

1. A medical device for approximating two mucosal surfaces comprising:
a device with a means for attaching the device to two mucosal surfaces simultaneously, thereby approximating the two mucosal surfaces.

2. A medical device for medializing a middle turbinate of a patient comprising:
a device with a means for attaching the device to the middle turbinate and the nasal septum of a patient simultaneously, thereby medializing the middle turbinate.

3. A medical device for medializing a middle turbinate of a patient comprising:
a device coated with a biocompatible adhesive suitable for adhering to the middle turbinate and the nasal septum, thereby medializing the middle turbinate.

4. A medical device for medializing a middle turbinate of a patient comprising:
a device with hooks, pins, barbs, or staples for attaching the device to the middle turbinate and the nasal septum of a patient simultaneously, thereby medializing the middle turbinate.

5. The device of claim 2, whereby medializing the middle turbinate prevents the middle turbinate from adhering to the lateral wall.

6. The device of claim 1,wherein the device is made of a bioabsorbable material.

7. The device of claim 6, wherein the material is selected from the group consisting of polyesters, polyanhydrides, polyamides, polycarbonates, polycarbamates, polyacrylates, polymethacrylates, polystyrenes, polyureas, polyethers, or polyamines.

8. The device of claim 1, wherein the device is made of a polyester.

9. The device of claim 1, wherein the device is made of PLGA.

10. The device of claim 1, wherein the device is made of a 85% D,L-lactide and 15% glycolide co-polymer; a 75% D,L-lactide and 25% glycolide co-polymer; 65% D,L-lactide and 35% glycolide co-polymer; or 50% D,L-lactide and 50% glycolide co-polymer.

11. The device of claim 3, wherein the adhesive is a cyanoacrylate adhesive.

12. The device of claim 3, wherein the adhesive is DERMABOND.

14. The device of claim 1, wherein the device is a wafer.

15. The device of claim 1, wherein the device is approximately 0.2 cm—approximately 2 cm in length and approximately 0.2 cm—approximately 2 cm in width.

16. The device of claim 1, wherein the device is approximately 1 cm by approximately 1 cm.

17. The device of claim 1, wherein the device is square, circular, oval, ring-shaped, rectangular, triangular, pentagonal, hexagonal, octagonal, zig-zag-shaped, or polygonal.

18. The device of claim 1, wherein the device is less than approximately 2 mm thick.

19. The device of claim 1, wherein the device is approximately 0.75 mm thick.

20. A medical device for medializing a middle turbinate of a patient comprising:
a sling for encircling or covering the middle turbinate and a means for attaching the sling to the nasal septum of a patient thereby medializing the middle turbinate.

21. A method of medializing the middle turbinate, the method comprising steps of: implanting a medical device between the middle turbinate and the nasal septum of a patient; and
adhering the middle turbinate to the nasal septum via the medical device.

21. The method of claim 20, wherein the medical device is the medical device of claim 1.

22. The method of claim 20, wherein the step of adhering comprises adhering the middle turbinate to the nasal septum for a sufficient time to allow the nose to heal after nasal surgery.

23. The method of claim 20, wherein the step of adhering comprises adhering the middle turbinate to the nasal septum for a time ranging from approximately 1 week to approximately 6 months.

24. The method of claim 20, wherein the step of adhering comprises adhering the middle turbinate to the nasal septum for a time ranging from approximately 3 weeks to approximately 8 weeks.

25. The method of claim 20, wherein the step of adhering comprises adhering the middle turbinate to the nasal septum for a time ranging from approximately 1 month to approximately 1 year.

26. The method of claim 20, wherein the step of adhering comprises using a cyanoacrylate adhesive and the medical device to attach the middle turbinate to the nasal septum.

27. A method of medializing the middle turbinate, the method comprising steps of:
adhering the middle turbinate to the nasal septum using an adhesive.

28. The method of claim 27, wherein the adhesive is a synthetic adhesive.

29. The method of claim 27, wherein the adhesive is a natural adhesive.

30. The method of claim 27, wherein the adhesive is a cyanoacrylate adhesive.

31. The method of claim 27, wherein the adhesive is a fibrin sealant.

32. The method of claim 27, wherein the adhesive is mussel adhesive protein or frog glue.

33. A method of medializing the middle turbinate, the method comprising steps of:
attaching the middle turbinate to the nasal septum using a pin.

34. A method of attaching the uvula to the nasopharyngeal side of the soft palate, the method comprising steps of:
attaching the uvula to the nasopharyngeal side of the soft palate of a subject using the medical device of claim 1.

35. An instrument for implanting the medical device of claim 1, comprising a means for holding the device and a means of compressing the nasal septum and middle turbinate around the device.

36. A kit comprising a medical device of claim 1.

37. The kit of claim 36 further comprising a device for implanting the device.

38. The kit of claim 36 further comprising instructions for using the device.

39. The kit of claim 36 further comprising a biocompatible adhesive.
